(19) United States
(12) Patent Application Publication
(10) Pub. No.: US 2024/0412839 A1
(43) Pub. Date: Dec. 12, 2024

(54) Dynamic Health Goal Monitoring For Wearable Device

(71) Applicant: Anhui Huami Information Technology Co., Ltd., Hefei (CN)

(72) Inventors: Mengfan Tang, Cupertino, CA (US); Matthew Laye, Cupertino, CA (US); Mingshu Shi, Hefei (CN); Kongqiao Wang, Hefei (CN)

(21) Appl. No.: 18/330,792

(22) Filed: Jun. 7, 2023

(51) Int. Cl. G16H 20/30; G06F 40/40; G16H 40/67; G16H 50/20; G16H 50/30

(52) U.S. Cl. CPC G16H 20/30; G06F 40/40; G16H 40/67; G16H 50/20; G16H 50/30

(57) ABSTRACT

A method of dynamically monitoring a health goal using a wearable device. The method may include receiving, by a processor, user input associated with the wearable device worn by an individual. The method may include obtaining, by the processor, health parameters associated with the individual from the wearable device and user feedback from the individual based on a physical condition of the individual associated with the health plan. Additionally, the method may include evaluating whether the health goal will be successfully reached by the individual following the health plan. Responsive to determining that the health goal will not be successfully reached, the health plan may be dynamically adjusted and provided to the individual to reach the health goal.

### TECHNICAL FIELD

This application relates to wearable computing, and in particular, dynamically adjusted training plans based upon large language model data and wearable computing input.

### BACKGROUND

Modern technologies have provided users with wearable computing devices configured to sense and track a user's physical parameters. Based upon such physical parameters of the user, the wearable computing devices of associated computer-based system may compute health-related analyses and recommendations to apply such information towards improved health of the user.

Current wearable computing devices may require an initial user input to set or determine a fitness goal, such a physical training to improve endurance and speed for a particular upcoming race or increased strength to improve overall physical output. Once goals have been set by the user, progress of the user towards the set goal cannot be tracked effectively, and the fitness goal cannot be adjusted during the course of training, resulting in a poor user experience.

### SUMMARY

Disclosed herein are implementations of methods, apparatuses, and systems for dynamic training plans.

In one aspect, a method of dynamically monitoring a health goal using a wearable device is disclosed. The method includes receiving, by a processor, user input associated with the wearable device worn by an individual to determine the health goal for the individual and a health plan associated with the health goal for the individual to reach the health goal; obtaining, by the processor, health parameters associated with the individual from the wearable device and user feedback from the individual based on a physical condition of the individual associated with the health plan; evaluating, by the processor based on the health parameters and the user feedback, whether the health goal will be successfully reached by the individual following the health plan; and responsive to determining that the health goal will not be successfully reached by the individual following the health plan, dynamically adjusting the health plan based upon the health parameters and the user feedback to determine a modified health plan, wherein the modified health plan is provided by the processor to the individual to reach the health goal.

In another aspect, an apparatus for dynamically monitoring a health goal using a wearable device is disclosed. The apparatus includes a non-transitory memory; and a processor configured to execute instructions stored in the non-transitory memory to: receive user input associated with the wearable device worn by an individual to determine the health goal for the individual and a health plan associated with the health goal for the individual to reach the health goal; obtain health parameters associated with the individual from the wearable device and user feedback from the individual based a physical condition of the individual associated with the health plan; evaluate, based on the health parameters and the user feedback, whether the health goal will be successfully reached by the individual following the health plan; and responsive to determining that the health goal will not be successfully reached by the individual following the health plan, dynamically adjust the health plan based upon the health parameters and the user feedback to determine a modified health plan and provide the modified health plan to the individual.

In another aspect, a non-transitory computer-readable storage medium configured to store computer programs for dynamically monitoring a health goal using a wearable device is disclosed. The computer programs include instructions executable by a process to: receive user input associated with the wearable device worn by an individual to determine the health goal for the individual and a health plan associated with the health goal for the individual to reach the health goal; obtain health parameters associated with the individual from the wearable device and user feedback from the individual based on a physical condition of the individual associated with the health plan; evaluate, based on the health parameters and the user feedback, whether the health goal will be successfully reached by the individual following the health plan; and responsive to determining that the health goal will not be successfully reached by the individual following the health plan, dynamically adjust the health plan based upon the health parameters and the user feedback to determine a modified health plan and provide the modified health plan to the individual.

### BRIEF DESCRIPTION OF DRAWINGS

FIG. 1 is a perspective view of an exemplary wearable device in accordance with the present teachings.

FIG. 2 is a block diagram of an example of a computing device that may be used with or incorporated into a wearable device in accordance with the present teachings.

FIG. 3 is a flowchart of an example process of dynamically monitoring a health goal using a wearable device in accordance with the present teachings.

FIG. 4 is a flow diagram of dynamic tracking and adjustment of an established training goal utilizing a wearable device in accordance with the present teachings.

FIG. 5 illustrates exemplary training types associated with a dynamic training plan.

FIG. 6 is a flow diagram illustrating weekly dynamic adjustment of an established training plan.

FIG. 7 is a flow diagram illustrating model training for intraweek dynamic adjustment of an established training plan.

FIG. 8 is a flow diagram illustrating exemplary user-agent interaction utilizing a dynamic large language model.

FIG. 9 illustrates exemplary user interfaces for a dynamic training plan with an associated flow diagram of the dynamic training plan process.

### DETAILED DESCRIPTION

Many portable devices and systems have been developed to monitor physiological conditions of an individual. One area of interest in the use of physiological monitors is personal wellness and physical exercise for purposes of fitness training, weight loss, or monitoring general health. This can include overall physical performance during fitness training, sleep duration, sleep interruptions, heart rate, and other physiological conditions. For example, physiological parameters of the individual can be continuously tested, including periodic recording of heart rate variability (HRV), to assess stress levels and physical fitness of the individual. The assessment may be carried out automatically during the individual's daily physical activities. Additionally, the assessment may allow for feedback from the individual regarding their own stress levels and physical fitness to enhance the overall assessment. Based on the assessment, various actions can be taken to keep the individual's health state.

In some instances, physiological conditions of the individual can be monitored and evaluated with respect to an established fitness goal. For example, the individual may establish an overall health goal and, as the individual completes daily physical activities to reach the overall health goal, the physiological conditions of the individual may be monitored and evaluated to determine if the individual is on track to successfully reach their overall health goal. If it is determined that the individual is unlikely to reach their overall health goal, the physiological conditions being monitored may be further analyzed to adjust the health goal or adjust a training plan associated with the health goal.

In some instances, physiological conditions of the individual can be monitored by, for example, displaying scores, such as physical stress levels of the individual, fatigue levels of the individual, or a recovery score, which gives the individual insights regarding their current physiological states. In additional to the physiological conditions monitoring, the individual may also provide feedback based upon the displayed scores to adjust or confirm the score determined, thereby providing a more accurate representation of the stress levels and physical fitness of the individual.

Using a data-driven approach, data from wearable devices such as smart watches can be analyzed to reveal what lifestyle patterns could positively or negatively affect an individual's ability to reach a health goal established by the individual. Implementations of this disclosure aim to help the individual successfully reach their health goal. For example, physiological condition data from the individual can provide indications about the individual's overall progress towards reaching their established health goal by following an associated health plan. As a result, the health plan being implemented may be adjusted to help the individual to reach the health goal.

According to implementations of this disclosure, a dynamic monitoring method is used to track progress of an individual using a health plan to reach their established health goal. The dynamic monitoring method may monitor physiological conditions of the individual, such as those mentioned above, to estimate the fitness progress of the individual compared to an expected progress level. For example, if the individual is following an established health plan to reach their health goal, the physiological conditions of the individual may be monitored and evaluated against an expected progress point or threshold associated with where the individual is expected to be at that given point in time. The individual may also provide subjective feedback as to their progress and physical stress levels, which may provide further data to help determine whether modifications to the health plan are necessary to still reach the health goal.

By dynamically monitoring physiological conditions of the individual and receiving feedback from the individual directly with respect to their physical fatigue or stress levels, a health plan associated with the individual's established health goal may be dynamically adjusted on a frequent and regular basis as needed. For example, evaluation of the individual's progress towards reaching their health goal may be actively evaluated throughout the individual's daily activities in addition or opposed to pre-established time periods set for evaluation. As a result, the health plan of the individual can be adjusted more frequently if needed to help the individual reach their health goal.

According to implementations of this disclosure, sensor data for the individual can be collected, for example, by wearable devices. The collected sensor data for the individual can be extracted to obtain various lifestyle and fitness data, including the heart rate or HRV data, sleep duration, sleep interruptions, body temperature, and other desired lifestyle data. Based upon the lifestyle and fitness data extracted, physical conditions and physical progress of the individual can be estimated. Based on one or more rules, the lifestyle and fitness data (e.g., heart rate data, sleep data, etc.) can be used to determine whether a health plan being followed by the individual requires adjustment in order to reach the established health goal.

To further refine the established health plan, a machine learning model may be trained using subjective feedback data from the individual and objective performance data collected from the wearable devices (e.g., the lifestyle data, the fitness data, etc.). The machine learning model may then be configured to actively monitor the health plan and adjust the health plan as needed based upon the data collected.

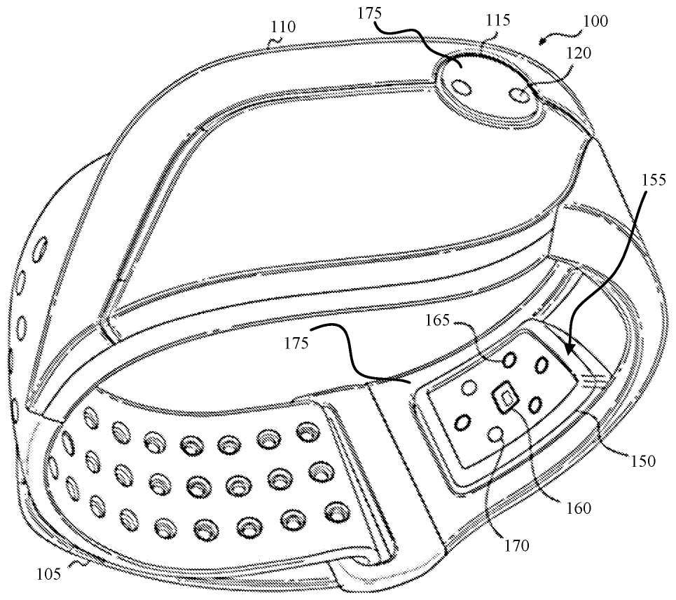

FIG. 1 depicts a perspective view of an exemplary device 100 according to some implementations of this disclosure. The device 100 may be a physiological monitor worn by an individual (also referred to herein as a user) to at least one of sense, collect, monitor, analyze, or display information pertaining to one or more physiological characteristics to provide information regarding health goal progress and the associated health plan established. The device 100 can include, for example, a band, a ring, a strap (e.g., a chest strap), or wristwatch. According to FIG. 1, the device 100 can include a wearable monitoring device configured for positioning at a user's wrist, arm, finger, chest, another extremity of the user, or some other area of the user's body.

The device 100 may include at least one of an upper module 110 or a lower module 150, each including at least one of one or more sensing tools. The sensing tools may include sensors and processing tools for detecting, collecting, processing, or displaying one or more physiological parameters and/or physiological characteristics of a user and/or other information that may or may not be related to health, wellness, exercise, sleep, or physical training sessions (e.g., characteristic information, education information, etc.).

The upper module 110 and the lower module 150 of the device 100 may include a strap or band 105 extending from opposite edges of each module for securing device 100 to the user. The band(s) 105 may include an elastomeric material or the band(s) 105 may include some other suitable material, including but not limited to, a fabric or metal material.

The upper module 110 or the lower module 150 may also include a display unit (not shown) for communicating information to the user (i.e., the wearer of the device). The display unit may be an LED indicator including a plurality of LEDs, each a different color. The LED indicator can be configured to illuminate in different colors depending on the information being conveyed. For example, where the device 100 is configured to monitor the user's heart rate, the display unit may illuminate light of a first color when the user's heart rate is in a first numerical range, illuminate light of a second color when the user's heart rate is in a second numerical range, and illuminate light of a third color when the user's heart rate is in a third numerical range. In this manner, a user may be able to detect his or her approximate heart rate at a glance, even when numerical heart rate information is not displayed at the display unit, and/or the user only sees the device 100 through the user's peripheral vision (e.g., while exercising).

The display unit may include a display screen for displaying images, characters, graphs, waveforms, or a combination thereof to the user or a medical professional. The display unit may further include one or more hard or soft buttons or switches configured to accept input by the user. Similarly, the display screen may be a touch screen configured to accept input by the user. The display unit may also switch or be toggled between displaying information.

The device 100 may further include one or more communication modules. Each of the upper module 110 and the lower module 150 may include a communication module such that information received at either module can be shared with the other module. One or more communication modules may also communicate with other devices such as a personal device of the user (such as a handheld device, a smart phone, a tablet, a laptop computer, a desktop computer, or the like) or a server (such as a cloud-based server). The communications between the upper and lower modules can be transmitted from one module to the other wirelessly (e.g., via Bluetooth, RF signal, Wi-Fi signal, near field communications, etc.) or through one or more electrical connections embedded in the band 105. Any analog information collected or analyzed by either module can be translated to digital information for reducing the size of information transfers between modules. Similarly, communications between either module and device can be transmitted wirelessly or through a wired connection and translated from analog to digital information to reduce the size of data transmissions.

As shown in FIG. 1, the lower module 150 can include an array of sensor array 155 including, but not limited to, one or more optical detectors 160, one or more light sources 165, one or more contact pressure/tonometry sensors 170, and at least one of the one or more gyroscopes or accelerometers 175. These sensors are only illustrative of the possibilities, however, and the lower module 150 may include additional or alternative sensors such as one or more acoustic sensors, electromagnetic sensors, ECG electrodes, bio impedance sensors, or galvanic skin response, or a combination thereof. Though not depicted in the view shown in FIG. 1, the upper module 110 may also include one or more such sensors and components on its inside surface (i.e., the surface in contact with the user's tissue or targeted area).

The location of the sensor array 155 or the location of one or more sensor components of the sensor array 155 with respect to the user's tissue may be customized to account for differences in body type across a group of users or placement in different locations on a user. For example, the band 105 may include an aperture or channel with which the lower module 150 is movably retained. In one implementation, the lower module 150 and the channel can be configured to allow the lower module 150 to slide along the length of the channel using, for example, a ridge and groove interface between the two components. For example, if the user desired to place one more component of the sensor array 155 at a particular location on his or her wrist, or mid-section, the lower module 150 can be slid into the desired location along the band 105. Though not depicted in FIG. 1, the band 105 and the upper module 110 can be similar configured to allow for flexible or customized placement of one or more sensor components of the upper module 110 with respect to the user's wrist or targeted tissue area.

The sensors and components proximate or in contact with the at least one of the user's tissue, the upper module 110, or the lower module 150 may include additional sensors or components on their respective outer surfaces (i.e., the surfaces facing outward or away from the user's tissue). In the implementation depicted in FIG. 1, the upper module 110 includes one such outward facing sensor array 115. The sensor array 115 may include one or more ECG electrodes 120, and/or one or more gyroscopes and/or accelerometers 175. Similar to the sensor arrays of the upper and lower modules proximate or in contact with the user's tissue, the outward facing sensor array 115 may further include one or more contact pressure/tonometry sensors, photo detectors, light sources, acoustic sensors, electromagnetic sensors, bio impedance sensors, accelerometers, gyroscopes, and/or galvanic skin response sensors.

The outward facing sensors of the sensor array 115 can be configured for activation when touched by the user (With his or her other hand) and used to collect additional information. The outward facing sensors may measure without being in direct contact with the user. The outward facing sensors of the sensor array 115 may be an accelerometer 175 and the accelerometer 175 may indirectly monitor movements or micro-movements (e.g., an acceleration or velocity change) that are transmitted to the sensor through the band or the module moving or being moved or a gyroscope that monitors velocities to determine micro-movements. In an example, where the lower module 150 includes one or more optical detectors 160 and light sources 165 for collecting ECG, PPG, or heart rate information of the user, the outward facing sensor array 155 of the upper module 110 may include ECG electrodes 120 that can be activated when the user places a fingertip in contact with the electrodes. While the optical detectors 160 and the light sources 165 of the lower module 150 can be used to continuously monitor blood flow of the user, the outward facing sensor array 115 of the upper module 110 can be used periodically or intermittently to collect potentially more accurate blood flow information which can be used to supplement or calibrate the measurements collected and analyzed by an inward facing sensor array, sensor array 155, or the lower module 150.

In addition to the inward and outward facing sensors, the device 100 may further include additional internal components such at least one of the one or more accelerometers or gyroscopic components for determining whether and to what extent the user is in motion (i.e., whether the user is walking, jogging, running, swimming, sitting, or sleeping), breathing rhythm, breathing signals, or a combination thereof of a user. Information collected by at least one of the accelerometer(s) or gyroscopic components can also be used to calculate the number of steps a user has taken over a period of time. The activity information may measurement movements. The movements measured may be macro-movements such as walking or jogging. The movements may by micro-movements.

The micro-movements may be caused by a surface of a user's skin or body part being moved due to, for example, respiration, heartbeat, or a combination thereof. The micro-movements may have a displacement (e.g., a length) less than a predetermined displacement in order for at least one of the accelerometer or gyroscope to at least of measure or record the micro-movements. For example, when a user walks the accelerometer may measure a movement of more than 1 cm, when the accelerometer detects a user heart beat the accelerometer may measure a displacement of between 4 mm and 1 cm (e.g., a micro-movement).

The displacement values and additional data collected from the sensor array 115 of the upper module 110 or the sensor array 155 of the lower module 150 may assist a non-transitory computer readable medium or processor in isolating various physiological conditions (e.g., heart beats, respiration, etc.). The processor may receive data from the sensor arrays 115, 155. The processor may dynamically filter the data. The process may analyze the data without regard to a position of the device relative to the user or a position of the user. The processor may filter unwanted signals and isolate only desired signals. For example, the processor may learn which signals are of interest and the process may analyze only those signals of interest. The processor may be in communication with or include a non-transitory computer-readable medium.

At least one of the upper or lower modules 110 or 150 can be configured to continuously collect data from a user using an inward facing sensor array. However, certain techniques can be employed to reduce power consumption and conserve battery life of the device 100. For instance, only one of the upper or lower modules 110 or 150 may continuously collect information. The module may be continuously active, but may wait to collect information when conditions are such that accurate readings are most likely.

For example, when one or more accelerometers or gyroscopic components of the device 100 indicate that a user is still, at rest, or sleeping, one or more sensors of at least one of the upper module 110 or the lower module 150 may collect information from the user while artifacts resulting from physical movement are absent. The accelerometer or gyroscope may not begin reading until the heart rate of the user measured by another sensor is below a predetermined limit. For example, if the ECG or PPG demonstrates that the user is moving, the accelerometer or gyroscope may not be turned on. In another example, the accelerometer or gyroscope may not begin reading until the heart rate of the user measured by another sensor is above a predetermined limit (e.g., the accelerometer or gyroscope only beings reading when a user is exercising).

The physiological information from the upper module 110, the lower module 150, or both may be graphically displayed or represented on a display (not shown) of the device 100. The graphical display may be provided as an output. The output may include physiological information of a user. For example, the information collected may be categorized and then graphically represented as one or more outputs. The output may include health plan or health goal data. For example, the output may graphically display progress towards an established health goal or display information regarding a currently established health plan associated with the health goal.

The output may also include education information pertaining to topics of interest for the user. For example, the display (not shown) may allow for user input to request information on specific topics or metrics. Based on such user input, the output may include information pertaining to the specific topics or metrics requested by the user.

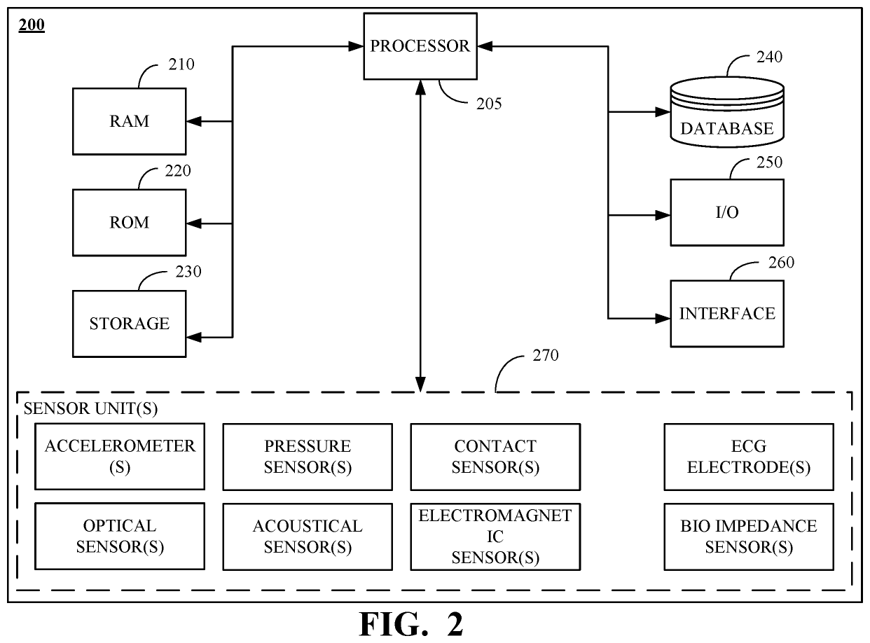

FIG. 2 depicts an illustrative processor-based computing device 200. The computing device 200 is representative of the type of computing device that may be present in or used in conjunction with at least some aspects of the device 100, or any other device comprising electronic circuitry. For example, the computing device 200 may be used in conjunction with any one or more of transmitting signals to and from the one or more optical sensors or acoustical sensors, sensing or detecting signals received by one or more sensors of the device 100, processing received signals from one or more components or modules of the device 100 or a secondary device, and storing, transmitting, or displaying information. The computing device 200 may be or may be included within the device 100. The computing device 200 may be a mobile terminal or remote device that is in communication with the device 100. The computing device 200, the device 100, or both may be in communication with a server (e.g., a cloud-based server). For example, the computing device 200 may be a separate device (e.g., a mobile terminal device) from the device 100, and both the computing device 200 and the device 100 may be in direct communication with the server. Alternative, the computing device 200 may be in direct communication with the server and the device 100 may be in communication with the serve view the computing device 200. It should also be noted that the computing device 200 is illustrative only and does not exclude the possibility of another process- or controller-based system being used in or with any of the aforementioned aspects of the device 100.

In one aspect, the computing device 200 may include one or more hardware and/or software components configured to execute software programs, such as software for obtaining, storing, processing, and analyzing signals, data, or both. For example, the computing device 200 may include one or more hardware components such as, for example, a processor 205, a random-access memory (RAM) 210, a read-only memory (ROM) 220, a storage 230, a database 240, one or more input/output (I/O) modules 250, an interface 250, and one or more sensor modules 270. Alternatively, and/or additionally, the computing device 200 may include one or more software components such as, for example, a computer-readable medium including computer-executable instructions for performing techniques or implement functions of tools consistent with certain disclosed embodiments. It is contemplated that one or more of the hardware components listed above may be implemented using software. For example, the storage 230 may include a software partition associated with one or more other hardware components of the computing device 200. The computing device 200 may include additional, fewer, and/or different components than those listed above. It is understood that the components listed above are illustrative only and not intended to be limiting or exclude suitable alternatives or additional components.

The processor 205 may include one or more processors, each configured to execute instructions and process data to perform one or more functions associated with the computing device 200. The term “processor,” as generally used herein, refers to any logic processing unit, such as one or more central processing units (CPUs), digital signal processors (DSPs), application specific integrated circuits (ASICs), field programmable gate arrays (FPGAs), and similar devices. As illustrated in FIG. 2, the processor 205 may be communicatively coupled to the RAM 210, the ROM 220, the storage 230, the database 240, the I/O module 250, the interface 250, and the one or more sensor modules 270. The processor 205 may be configured to execute sequences of computer program instructions to perform various processes, which will be described in detail below. The computer program instructions may be loaded into the RAM 210 for execution by the processor 205.

The RAM 210 and the ROM 220 may each include one or more devices for storing information associated with an operation of the computing device 200 and/or the processor 205. For example, the ROM 220, may include a memory device configured to access and store information associated with the computing device 200, including information for identifying, initializing, and monitoring the operation of one or more components and subsystems of the computing device 200. The RAM 210 may include a memory device for storing data associated with one or more operations of the processor 205. For example, the ROM 220 may load instructions into the RAM 210 for execution by the processor 205.

The storage 230 may include any type of storage device configured to store information that the processor 205 may use to perform processes consistent with the disclosed embodiments.

The database 240 may include one or more software and/or hardware components that cooperate to store, organize, filter, and/or arrange data used by the computing device 200 and/or the processor 205. For example, the database 240 may include user profile information, historical activity and user-specific information, physiological parameter information, predetermined menu/display options, and other user preferences. Alternatively, the database 240 may store additional and/or different information. For example, the database 240 may include information to establish a large language model (LLM) of the I/O module 250.

The I/O module 250 may include one or more components configured to communicate information with a user associated with the computing device 200. For example, the I/O module 250 may include one or more buttons, switches, or touchscreens to allow a user to input parameters associated with the computing device 200. The I/O module 250 may also include a display including a graphical user interface (GUI) and/or one or more light sources for outputting information to the user. The I/O module 250 may also include one or more communication channels for connecting the computing device 200 to one or more secondary or peripheral devices such as, for example, a desktop computer, a laptop, a tablet, a smart phone, a flash drive, or a printer, to allow a user to input data to or output data from the computing device 200.

The interface 260 may include one or more components configured to transmit and receive data via a communication network, such as the internet, a local area network, a workstation peer-to-peer network, a direct link network, a wireless network, or any other suitable communication channel. For example, the interface 260 may include one or more modulators, demodulators, multiplexers, demultiplexers, network communication devices, wireless devices, antennas, modems, and any other type of device configured to enable data communication via a communication network.

The computing device 200 may further include the one or more sensor modules 270. In one embodiment, the one or more sensor modules 270 may include one or more of an accelerometer module, an optical sensor module, an acoustical sensor module, and/or an ambient light sensor module. It should be noted that these sensors are only illustrative of a few possibilities and the one or more sensor modules 270 may include alternative or additional sensor modules suitable for use in the device 100. It should also be noted that although one or more sensor modules are described collectively as the one or more sensor modules 270, any one or more sensors or sensor modules within the device 100 may operate independently of any one or more other sensors of sensor modules. Moreover, in addition to collecting, transmitting, and receiving signals or information to and from the one or more sensor modules 270 at the processor 205, any of the one or more sensors of the one or more sensor modules 270 may be configured to collect, transmit, or receive signals or information to and from other components or modules of the computing device 200, including but not limited to the database 240, the I/O module 250, or the interface 260.

As described above with respect to FIG. 1, the one or more accelerometers of the device 100 can be used to detect large-scale motions of a subject indicative of physical activity (e.g., steps, running, walking swimming, etc.) The same accelerometers can be used to determine the onset of a sleep period through the detection of a lack of motion. The one or more acoustical sensors can be used to detect and monitor heart rate. However, the sensitivity of the acoustical sensor(s) that detect heart rate aren't sensitive enough to detect relatively slow heart rate during sleeping. In one embodiment, upon determining that the subject is engaged in sleep, the sensitivity of the acoustical sensor(s) can be reconfigured to detect a significantly lower heart rate. Alternatively, the device 100 may include one or more acoustical sensors that are dedicated to, and configured for, detecting relatively slow heart rate during sleeping while one or more other acoustical sensors are used to detect regular heart rate during physical activity.

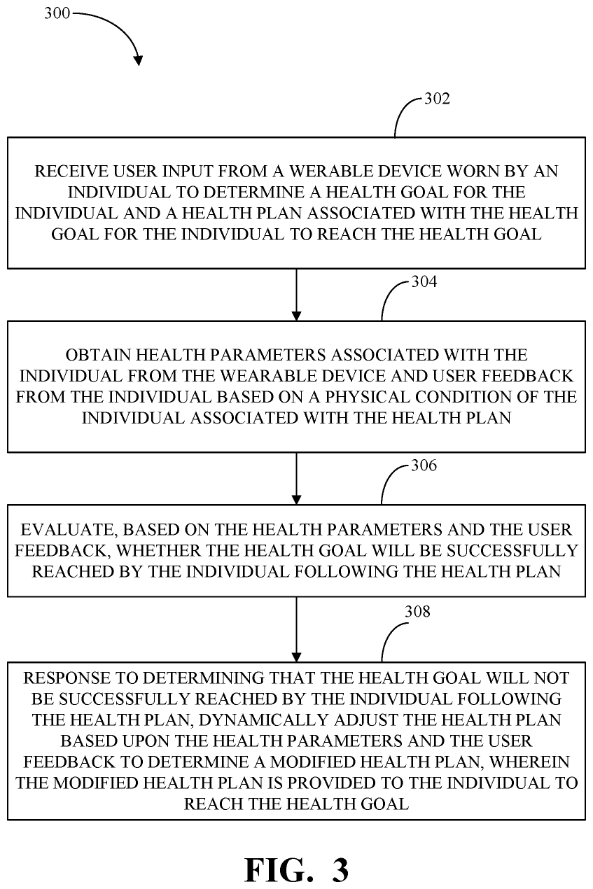

FIG. 3 is a flowchart of an example process 300 of dynamically monitoring a health goal using a wearable device according to some implementations of this disclosure. It should be noted that flowchart and the process 300 may be used interchangeably herein. The process 300 can be implemented as software and/or hardware modules in the computing device 200 of FIG. 2. For example, the process 300 can be implemented as software modules stored in the storage 230 as instructions and/or data executable by the processor 205 of an apparatus, such as the device 100 in FIG. 1. In another example, the process 300 can be implemented in hard as a specialized chip storing instructions executable by the specialized chip. Some or all of the operations of the process 300 can be implemented by the processor 205 of FIG. 2. As described above, a person skilled in the art will note that all or a portion of the aspects of the disclosure described herein can be implemented using a general-purpose computer/processor with a computer program that, when executed, carries out any of the respective techniques, algorithms, and/or instructions described herein. In addition, or alternatively, for example, a special-purpose computer/processor, which can contain specialized hardware for carrying out any of the techniques, algorithms, or instructions described herein, can be utilized.

Similarly, all or a portion of the aspects of the disclosure described herein can be implemented by the device 100 (e.g., by the processor 205 when the computing device 200 is incorporated into the device 100), by a server in communication with the device 100 and/or the computing device 200, or both. Additionally, all or a portion of the aspects of the disclosure described herein (e.g., steps, procedures, processes, etc.) may be performed by the device 100 or a secondary companion device (e.g., a mobile terminal, a client device, other remote device, etc.). For example, a portion of the steps or procedures described herein may be performed by the aforementioned server while another portion of the steps or procedures may be performed by the secondary companion device.

At an operation 302, user input data from an individual can be obtained to determine a health goal for the individual. The health goal may be a target goal for the individual to reach over a set period of time. For example, the health goal may be a target running distance for an upcoming race or a target completion time for an upcoming race. Alternatively, the health goal may be a target weight for a particular exercise of strength training or even a more general physiological goal, such as a target weight loss or weight gain goal.

At the operation 302, the user input data may be received through a device, such as the device 100 shown in FIG. 1. For example, the device may include an interactive display that prompts a user to enter specific health goal parameters. The user input data may be received through a companion or secondary device, such as a client device or terminal device (e.g., mobile device, computer, etc.). For example, the user input data may be received through a terminal or remote device, in which the terminal or remote device may include an application that provides a user interface to the user to enter user input data.

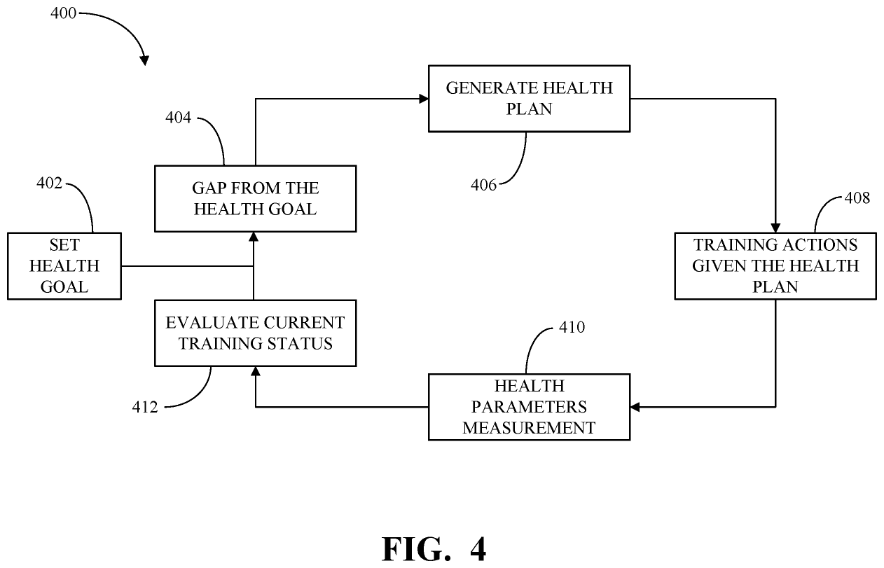

In one example, the user may want to establish a health goal for reaching a certain distance of running in a desired duration of training time. To establish such a goal, the user may be prompted to input specific parameters, such as but not limited to, a target running distance, a date of an upcoming race or target completion date for training, and a level of running experience (e.g., an advanced runner, a new runner, etc.). After the user inputs the requisite parameters, the operation 302 may establish a correlated health goal based on the user inputs. Additionally, in order for the individual to reach the established health goal, a health plan will also be established to provide the user with guidance to successfully reach their health goal. For example, if a target running distance for an upcoming race is established as the health goal, the health plan may provide the user an exercise training schedule for the user to reach a fitness level needed to achieve the health goal. Health goal and health plan establishments are explained in further detail below with respect to FIG. 4.

After the operation 302 and at an operation 304, health parameters associated with the individual may be obtained by data collected by the device 100 of FIG. 1, such as a smart watch, for example. Data collected by the device 100 can be obtained, for example, by the processor 205. The data collected by the device 100 can be physiological information such as, for example, a heart rate of the individual during or outside of exercise, sleep duration and/or sleep apnea conditions of the individual, body temperature, or other physiological information.

Depending on the particular health goal and health plan determined at operation 302, different health parameters associated with the individual may be obtained. That is, different health goals and health plans may require different health parameter data to be collected. For example, if a health goal established by the individual is a target race duration, the health parameters collected may include the individual's heart rate and running speed (acceleration and/or velocity). In another example, if a health goal established is a target strength training weight, the health parameters collected may include the individual's heart rate, sleep duration and/or sleep apnea conditions, and body temperature. However, the health parameters collected can be any combination of health parameters possible.

In addition to health parameters, user feedback may also be collected by the device 100, such as through a user interface of the device 100. The user interface can be a display or physical buttons of the device 100 that facilitate a user to input their feedback into the device. Alternatively, the user interface may allow for the user to verbally communicate their feedback using one or more audio components of the device (e.g., a microphone, speaker, audio amplifier, audio codec, etc.). The device 100 may prompt the user for feedback regarding health parameters received by the device 100 or may prompt the user for additional feedback regarding other physiological conditions.

In one example, at the operation 304, the individual may provide user feedback based on his or her physical condition. The individual's physical condition may be associated with activities completed in accordance with the health plan established. For example, the user may provide feedback on his or her physical condition after completing a particular exercise within the health plan. However, in certain embodiments, the individual may provide user feedback at any desired time at their own discretion.

As stated above, the user feedback may be associated with a physical condition of the individual in association with the established health plan. The physical condition may be a level of inflammation of the individual, a level of fatigue, an injury of the individual, other medical conditions of the individual, or a combination thereof. Additionally, the physical condition may be a subjective determine by the individual whether a particular aspect of the health plan was challenging for the individual of whether the individual believed the particular aspect of the health plan was completed with ease. However, the user feedback received at the operation 304 need not be limited to any particular feedback, and in various embodiments, the feedback may reflect any number of different parameters, physiological conditions, mental conditions (e.g., mental fatigue, stress, anxiety, etc.), or a combination thereof.

Once the health parameters and the user feedback are obtained at the operation 304, at an operation 306 the health parameters and the user feedback may be evaluated to determine whether the established health goal will be successfully reached by the individual following the established health plan. That is, evaluation at the operation 304 may determine whether the individual will successfully reach the health goal if the health plan were to remain the same with no modifications. The evaluation at the operation 304 may be completed by executing a variety of instructions or modules utilizing the processor 205. For example, the processor 205 may execute one or more software modules stored in the storage 230 to evaluate whether the health goal will be met. Such software modules being executed by the processor 205 may require the health parameters and/or user feedback obtained as inputs for evaluation. Such evaluation will be discussed in further detail below.

If the evaluation at the operation 304 determines that the health goal will be successfully reached if the individual maintains training based on the health plan, no adjustments to the health plan or health goal may be needed. However, if the evaluation at the operation 304 determines that the health goal will not be successfully reach by the individual following the health plan, a response may be needed at operation 308.

At the operation 308, the response may be to dynamically adjust the health plan initially established at the operation 302. However, it should also be noted that the response at the operation 308 may also dynamically adjust an intermediate health plan established after the initial health plan. In other words, give the dynamic nature of monitoring the health goal and the associated health plan, the process 300 may be completed any number of times before, during, or after execution of the health plan. Furthermore, while modification at the operation 308 is being done to the health plan, in certain circumstances modification may also, or alternatively, be done to the health goal. For example, if the evaluation at operation 306 determines that the health goal is unattainable based on the health parameters and the user feedback obtained, the individual may be prompted to modify their health goal or, alternatively, the device 100 implementing the process 300 may establish a modified health goal and prompt the individual for approval of the modified health goal.

Dynamically adjusting the health plan at the operation 308 may be completed based upon the health parameters and the user feedback. For example, the health parameters may indicate that the individual has exercised at a level below an exercise level of the health plan at a particular time. Similarly, the health parameters and/or the user feedback may indicate that the individual has slept poorly for an extended period of time or has increased inflammation and, as a result, the individual may likely have increased fatigue levels that hinder successfully maintaining the current health plan without an extended rest and/or recovery incorporated into the health plan. As a result, the health plan may be modified to account for such factors and be provided to the individual (e.g., through the device 100 or a display thereof) to correct a course of training to still meet the established health goal.

FIG. 4 illustrates a flow diagram 400 of dynamic tracking and adjustment of an established training goal utilizing a wearable device, such as the device 100 in FIG. 1. The flow diagram 400 may be used interchangeably with the process 400 as detailed below. Initially, a training goal may be set at operation 402. The operation 402 may be completed at operation 302 of the process in FIG. 3. However, in certain embodiments, the health goal set at operation 402 may be completed after the initial health goal established at the operation 302. For example, the health goal may be modified or reestablished if the individual wishes to change their health goal.

As mentioned above, the health goal may be set based on user input from the individual. The user input may be provided through the device 100, such as a display of a smart watch. The device 100 may prompt the user to input particular answers to specified questions or may provide the user with selectable options to set the health goal. For example, the user (i.e., the individual) may be prompted to provide their desired health goal as well as their current status with respect to achieving the health goal. While a user may be prompted to more generally input a desired health goal, in some embodiments the operation 402 may request or require the user to enter specific parameters to set the health goal. The parameters may vary depending on the type of health goal. The health goal may be an exercise activity goal, such as a running goal or swimming goal, or the health goal may be an overall fitness goal, such as a weight loss goal or a strength training goal. As a result, the parameters requested for input may vary to reflect a particular type of health goal. Therefore, in certain embodiments, the initial user input may be to select a particular type of health goal (e.g., running, swimming, strength training, weight loss, etc.) before additional, more specific parameters are requested. For example, if a running or swimming goal were to be selected initially by the individual, the individual may be prompted for more specific inputs that may include, but are not limited to, a target distance, a race or competition date (if applicable, or alternatively, a target end date for training), a current level of skill in that particular type of training, any current health conditions that may pose a risk and/or hinderance to training, any current disabilities that prevent certain types of training, or a combination thereof.

In addition to user input regarding the type of health goal, the operation 402 may also utilize more general physiological data input from the user to establish the health goal. The more general physiological data may include, but is not limited to, gender of the individual, age of the individual, height of the individual, weight of the individual, or a combination thereof.

Furthermore, the user may also input a current status with respect to the desired health goal. For example, if the individual is establishing a target running distance and/or running time, the device 100 may prompt the user to input a current running pace (i.e., a time of completion for a specific distance)

Based upon the aforementioned user input, the initial health goal with be set at the operation 402. After setting the health goal, a gap between the current status of the individual and the health goal may be determined at operation 404. The gap may be a pace gap if completing a distance-related health goal, such as running, biking, swimming, etc. The gap may be a weight gap if completing a strength training or weight-related health goal.

For illustrative purposes, in one particular embodiment, a health goal may be set at the operation 402 by the individual for a target completion time for a specific running distance for an upcoming race. A gap from the health goal established at the operation 404 may then be a pace gap. That is, the health goal may correlate to a running pace goal while the current status of the user may correlate to a current running pace of the individual. The pace goal (i.e., the health goal) may be determined using the following:

$$PACE GOAL = \frac{DISTANCE}{COMPLETION TIME GOAL} \qquad ( Equation 1 )$$

wherein DISTANCE represents a target running distance of the individual, and COMPLETION TIME GOAL represents a target completion time for completing the target running distance.

The pace goal (i.e., the health goal) may then be calculated and set at the operation 402. Based on the pace goal, the pace gap may be determined at the operation 404 using the following:

$$PACE GAP = CURRENT PA CE - PACE GOAL \qquad ( Equation 2 )$$

wherein PACE GAP represents a time gap between the PACE GOAL as determined above, and CURRENT PACE represents a currently measured running pace of the individual using, for example, the device 100.

By way of example, the individual may be prompted by the device 100 to input a target distance and a desired completion time goal for the target distance. The PACE GOAL may then be determined using Equation 1 above, wherein the DISTANCE is the target distance input by the individual and the COMPLETION TIME GOAL is the desired completion time goal input by the individual. Additionally, a CURRENT PACE may be determined based upon performance of the individual, for example, by the wearable device based on at least one measurement, which measures a time for the individual to physically run a measured distance. Once the CURRENT PACE has been determined, the PACE GAP may be determined using Equation 2 above, whereby the PACE GAP may be determined as the difference between the CURRENT PACE as measured and the PACE GOAL calculated.

Once the pace gap or other gap is determined at the operation 404, a health plan may be generated at operation 406. The health plan may be associated with the health goal to provide the individual guidance as to how to accomplish the health goal. In particular, the health plan may be generated or established to aid the individual with shortening the pace gap or other gap between the individual's current pace (i.e., current status) and the established health goal.

The health plan may be a training plan following by the individual. The training plan may include one or more training activities or actions needing to be completed by the individual to achieve his or her established health goal. The training plan may be generated based upon the initial user feedback provided by the individual (e.g., target distance, race date or target completion date, due date, current level of skill, etc.) and/or based upon the pace gap determine in the operation 404.

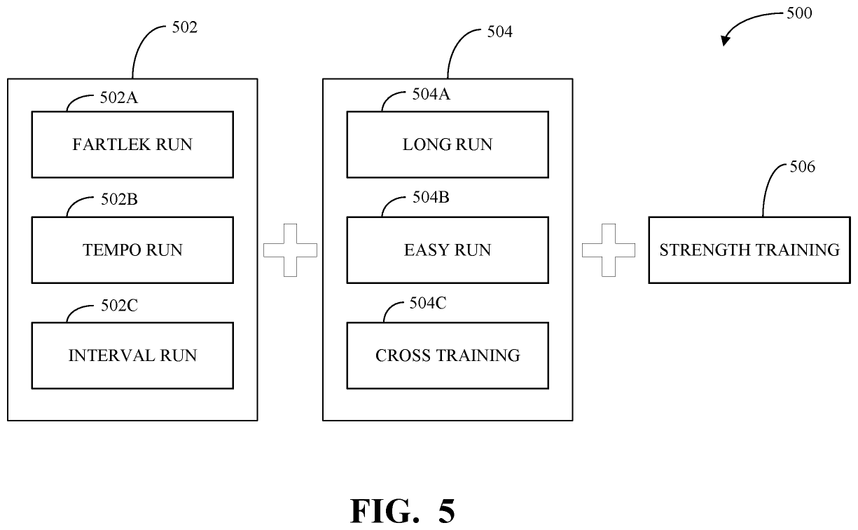

By way of example, the initial training plan may be generated based on the following:

$$INITIAL TRAINING PLAN = {f}_{1} ( PG , TD , RD , L ) \qquad ( Equation 3 )$$

where PG is the estimated or calculated pace gap, TD is the target distance, RD is the racing day, and L is the level of skill of the individual. Additionally, f₁ may be a function of the pace gap, target distance, racing day, and level of skill of the individual based on user behavior data and knowledge of sports science pertaining to various running activity components. For example, user behavior data may include subjective data provided by the individual (e.g., the initial user input) and/or objective data provided from other data sources as a baseline or benchmark. As a result, the initial training plan (i.e., the health plan) generated at the operation 406 may include various components or activities within specified performance improvement categories, which are further discussed with respect to FIG. 5.

For illustrative purposes, the individual may be prompted by the device 100 to input a target running distance (TD), a date of their upcoming race (RD), and an experience level in running (L) at the operation 402. Additionally, the pace gap (PG) may be determined using Equation 2, as described above with respect to the operation 404. From here, the INITIAL TRAINING PLAN may be determined for the individual as a function of the input data (e.g., the targeting running distance (TD), the upcoming race date (RD), and the experience level in running (L)) and the calculated pace gap (PG). As a result, the INITIAL TRAINING PLAN may provide a training plan to the individual specifically tailored to the individual and their needs, in which the training plan may include specific exercises or activities to achieve the individual's health goal.

Once the health plan is generated in the operation 406, training actions may be provided to the individual according to the generated health plan. For example, specified exercises, runs, strength training regiments, sleep schedules, general diet recommendations, other actions, or a combination thereof may be provided to the individual to complete the training plan. Such training actions may be provided incrementally or in various manners (e.g., through the device 100 or a display thereof) so that the individual may follow the health plan. The training actions may be associated with specified dates or times of the health plan or may be provided to the individual based on specified thresholds. For example, certain training actions may only be provided to the individual after the individual reaches a particular training threshold, such as a threshold pace or distance. In another example, certain training actions may continue to be provided to the individual until the individual reaches a particular training threshold, after which the individual may no longer be provided those training activities. For another example, certain training actions for a period or sub-period of time are provided to the individual before or at the beginning of the period or sub-period of time, and the period or sub-period of time may be one day, one week or the like. However, one skilled in the art would understand that various training plan iterations may be possible based upon the present teachings.

After the training plans are provided to the individual at the operation 408, health parameters of the individual may be measured at operation 410. The health parameters may be measured with the device 100 as discussed above and may include any of the aforementioned health parameters (e.g., heart rate, body temperature, sleep duration, etc.). The health parameters may be actively measured before, during, or after training actions are completed by the individual. For example, the health parameters may be measured during physical exertion while completing the training actions provide and/or the health parameters may be measured before or after physical exertion, such as during periods of rest and/or sleep.

Based upon the health parameters measured at the operation 410, a current training status may be evaluated at operation 412. Evaluation of a current training status of the individual may be completed dynamically in real-time to take into account the actively measured health parameters, user feedback provided by the individual, or both. Evaluation may therefore beneficially be utilized dynamically adjust the training plan as needed. For example, as discussed with respect to the process 300 in FIG. 3, evaluation of the current training status may include evaluating whether the established health goal from at the operation 402 will be successfully reached if the individual were to maintain the current health plan. If the current training status is determined to be acceptable or well followed by the individual, no changes to the health plan (i.e., training plan) may be needed. However, if the training status is determined to be unacceptable or poorly followed by the individual, the health plan may need to be adjusted according to the operation 308.

After evaluation of the current training status is completed at the operation 412, the gap to the health goal may be recalculated at the operation 404 to determine if the gap has been shortened based upon the initial training actions of the health plan being completed. As a result, the process 400 may be repeated actively to continue to decrease the gap between the current pace of the individual and the pace goal (i.e., the health goal) until the health goal is achieved. Additionally, as the process 400 is repeated and additional data is collected from the health parameters and/or user feedback, training health plan may continue to be adjusted as needed or more accurately fine-tuned in order for the individual to reach the health goal. Such dynamic adjustment of the training plan will be discussed in further detail below.

FIG. 5 illustrates exemplary training categories for the health plan (i.e., training plan) generated at the operation 406 shown in FIG. 4. As discussed above with respect to FIG. 4, the initial training plan may be established based on one or more initial parameters collected from the individual. The training plan may also, or alternatively, be based on user behavior data of the individual or general behavior data collected and stored in a database along with data pertaining to sports science. For example, the training plan generated may provide training actions at operation 408 which are split into specific categories based on the behavior data and/or the sports science data. The categories may focus on performance improvement in particular areas such as, but not limited to, anaerobic capacity of the individual, aerobic capacity of the individual, and body strength of the individual. As such, training actions provided to the individual may be categorized as anaerobic capacity activities 502, aerobic capacity activities 504, and body strength activities 506, as shown in FIG. 5.

Each of the activity categories may include one or more specific activities (e.g., exercises) to help improve performance of the individual's capacity in that particular area. FIG. 5 illustrates exemplary activities, though it should be noted that other activities not shown may be included in each of the categories. As shown in FIG. 5, anaerobic capacity activities 502 may include, but is not limited to, fartlek runs 502A, tempo runs 502B, and interval runs 502C. Aerobic capacity activities 504 may include, but is not limited to, long runs 504A, easy runs 504B, and cross training 504C. Body strength activities 506 may be various strength training exercises. The specifics of each activity provided to the individual may be tailored to his or her specific health goal and current training status evaluated at the operation 412.

Advantageously, the health plan established may provide the individual with varied training actions to improve both strength and conditioning, thereby providing an improved foundation to meet the health goal.

In some implementations, the time duration of the training plan, such as from the beginning to the end of the training plan or from the time when the training plan is established to the due race date, can be divided into at least one period of time, and the training plan for the health goal may include a training plan for each of the at least one period of time. A period of time may be one month, one week, one day or any time duration depending on the time duration of the training plan, or a period of time is a preset time duration. For example, the training plan for a period of time may include an overall amount of training to be completed during the period, a target for shortening the gap to the health goal, or both. The overall amount of training to be completed may include accumulated amount of exercise, accumulated impulse value, accumulated training time duration, accumulated training time duration for one specific training intensity, or any combination of these. For another example, a period of time can be divided into a plurality of sub-periods, and the training plan for a period of time may include training plans of each of the plurality of sub-periods. For instance, a weekly training plan includes a training plan for each day of the week that outlines whether to rest or train, the duration of each training session, the intensity of each training session, training categories, specific exercises to do, or any combination thereof. One example of the training plan for a week is shown in table 1.

TABLE 1

| | | Estimated | | |
|---|---|---|---|---|
| Week- | Training | duration | Estimated | |
| day | type | (min) | intensity | Details |
| Mon | Run - | 60 | 1 | 55-65 Minutes Easy Run |
| | Long Run | | | |
| Tue | Run - | 62 | 1.5 | Warm up: 15 minutes Z1 |
| | Intervals | | | Intervals: 8 × 2 minutes |
| | | | | @100% of 5 k goal pace, |
| | | | | 2 minutes @60% |
| | | | | of 5k goal pace |
| | | | | Cool down: 15 minutes Z1 |
| Wed | Run - Easy | 35 | 1 | 30-40 Minutes Easy Run |
| Thu | Run - Easy | 35 | 1 | 30-40 Minutes Easy Run |
| Fri | Off | 0 | 0 | Rest |
| Sat | Off | 0 | 0 | Rest |
| Sun | Off | 0 | 0 | Rest |

The health plan can be dynamically adjusted periodically. For example, the health plan is dynamically adjusted based on a period, and during or after a period of time, it is determined whether to adjust the training plan for the next period of time or the training plan for the rest of the training course based at least on the health parameters measured during that period of time. Alternatively, the health plan is dynamically adjusted based on a sub-period, and during or after a sub-period of time, it is determined whether to adjust the training plan for the next sub-period based at least on the health parameters measured during this sub-period. For yet another example, the training plan is dynamically adjusted based on both a period and a sub-period. In an instance, a period is a week, a sub-period is a day, and the training plan for a week includes training plans for every day of the week. The training plan of a week can be dynamically adjusted before or during the week based at least in part on the health parameters of the individual in the last week, so as to obtain the adjusted training plan of the week, e.g., the adjusted training plan of each day of the week. Furthermore, before or during each day, the adjusted training plan of the day can be dynamically adjusted based at least in part on the health parameters of the individual before this day of the week, to obtain a further adjusted training plan of the day.

The health plan can be dynamically adjusted in response to detecting the occurrence of a preset condition. The preset condition may be triggered by receiving a preset input command, determining that the individual has deviated from the training plan beyond a certain range, determining that the individual's progress towards the health goal does not meet a preset threshold, or any other condition set by the user.

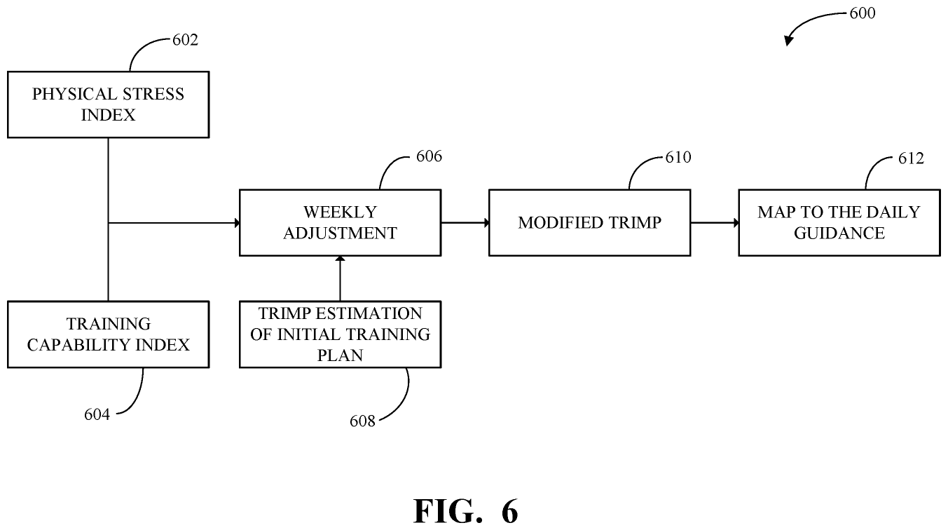

FIG. 6 illustrates a flow diagram 600 for weekly adjustment of the health plan (i.e., training plan) established at the operation 406. The flow diagram 600 may be used interchangeably with the process 600 as detailed below. The weekly adjustment shown in FIG. 6 may be completed at the operation 308 of the process 300 shown in FIG. 3. As discussed above, the health plan generated may be dynamically modified based on user feedback and one or more health parameters measured by the device 100 of FIG. 1. Modifications to the health plan may be done in the short-term, in the long-term, or both. For example, short-term adjustments of the health plan may be done daily or in increments of less than one day as needed. Long-term adjustments may be done on a weekly basis. However, it should be noted that short-term modifications or adjustments to the health plan may be done incrementally in a duration of time that is less than the duration of time for long-term modifications or adjustments (i.e., daily or even hourly for short-term adjustments to the health plan and weekly or bi-weekly adjustments for long-term adjustments to the health plan).

Turning now back to FIG. 6, the flow diagram 600 may be for long-term adjustments on a weekly basis as needed. To evaluate and determine whether weekly adjustment to the health plan is needed, a physical stress index at operation 602 and a training capability index at operation 604 may be estimated or calculated.

The physical stress index (PSI) may be a parameter that estimates the individual's acute training load (ATL) compared to the chronic training load (CTL) of the individual. To calculate the PSI, a daily training load may be estimated based on a training impulse (TRIMP) of the individual. TRIMP is a weighted product of training volume and training intensity over a duration of time. TRIMP may be calculated as:

$$TRIMP = {\sum }_{t = 1}^{T} HRL ( t ) \qquad ( Equation 4 )$$

where HRL is the heart rate level at a designated time t. The exercising heart rate (HR) of the individual may be converted into the HRL by, for example, implementing a stepwise function of HR and a maximum heart rate (MHR) of the individual. An example output HRL for a stepwise function as described above is as follows:

```
IF HR < 0.55 * MHR:
  RETURN 0
IF HR < 0.7 * MHR:
  RETURN 1
IF HR < 0.8 * MHR:
  RETURN 2
ELSE:
  RETURN 3
```

The MHR of the individual may be estimated by an age formula using the individuals age, such as MHR=220−age. However, to more accurately determine the physical stress index at the operation 602, the individual may also be prompted or have the option to input their MHR data manually (e.g., via the device 100).

For example, the device 100 may prompt the individual to input their age to estimate their MHR (e.g., MHR=220−age) or manually input their known MHR. The device 100 may then measure (e.g., by one or more sensors) the individual's heart rate during exercise (HR) at specified times (t), such as when following the established training plan. The measured heart rate (HR) may then be compared to the MHR utilizing a stepwise function such as the stepwise function outlined above. Based on the stepwise function, a value may be determined for the individual's HRL at the time of each measurement (t) by the device 100. The TRIMP may then be determined using Equation 3 based upon the HRL values.

Once the HRL and TRIMP are calculated based on the above, the PSI may be estimated by the accumulation of TRIMP in the short-term (D1) and the long-term (D2) as follows:

$$PSI = \frac{{\sum }_{d - D 1}^{d} TRIMP ( d )}{{\sum }_{d - D 2}^{d} TRIMP ( d )} \qquad ( Equation 5 )$$

TRIMP may be determined based on Equation 4, as outlined in the above example. To determine the PSI, the short-term duration (D1) and the long-term duration (D2) may be established as specified accumulation durations. For example, the short-term duration (D1) may be used for accumulation of TRIMP over the course of a day, whereas the long-term duration (D2) may be used for accumulation of TRIMP over the course of a week.

After calculation based on the above, the PSI may be evaluated based on pre-defined thresholds to determine a training status of the individual. That is, the PSI estimated at operation 602 may be categorized based on the pre-defined thresholds to aid in determining whether weekly adjustment at the operation 606 is needed. By way of example, the following may be the pre-defined thresholds:
PSI value less than 0.5=Detraining, whereby the PSI indicates that the individual is getting less fit and is lacking exercise.
PSI value between 0.5 and 0.9=Maintaining, whereby the PSI suggests that the individual is maintaining the current training load based on the health plan.
PSI value between 0.9 and 1.4=Improving, whereby the PSI suggests that the individual has acceptable stress on his or her body, which may lead to adaptation and improvement based on the health plan.
PSI value over 1.4=Over-trained, whereby the PSI suggests that the individual has excessively trained, which may increase injury risk to the individual.

Based on the above categories, the PSI may be assigned as a factor in the weekly adjustment evaluation completed at the operation 606. In addition to the PSI, the training capability index may be estimated at the operation 604. The TCI may estimate how the individual followed the health plan (i.e., training plan) in given week w prior to any potential weekly adjustments done at the operation 606. However, it should be noted that the TCI may be adjusted for estimated how the individual followed the health plan other durations of time, such as bi-weekly, monthly, etc. The TCI can be calculated as:

$$TCI = \frac{{Actual\_TRIMP}_{w}}{{Expected\_TRIMP}_{w}} \qquad ( Equation 6 )$$

where Actual_TRIMP_{w} may be calculated based on Equation 4 and Expected_TRIMP_{w} may be estimated as a projected TRIMP for the individual for the given week w. For example, the Expected_TRIMP_{w} may be estimated based on the actual TRIMP values determined (e.g., by Equation 4) for a previous week and expectations of the individual maintaining a similar progression.

Similar to the PSI, the TCI may be evaluated based on pre-defined thresholds to determine a training capability of the individual. That is, the TCI estimated at the operation 604 may be categorized based on the pre-defined thresholds to aid in determining whether weekly adjustment at the operation 606 is needed. By way of example, the following may be the pre-defined thresholds:
TCI value greater than 1.4=Over-motivated, whereby the TCI suggests that the individual is overly motivated and exercising too frequently and/or too aggressively.
TCI value between 0.6 and 1.4=Optimal, whereby the TCI suggests that the individual is training in an optimal manner.
TCI value less than 0.6=Demotivated, whereby the TCI suggests that the individual not exercising sufficiently.

Once the PSI and TCI values are estimated, both values may be evaluated for potential weekly adjustment to the health plan at the operation 606. For evaluation at the operation 606 using the PSI and TCI values, a TRIMP adjustment ratio (Adj_ratio) can be applied with PSI and TCI inputs, as:

$${Adj}_{ratio} = {f}_{2} ( PSI , TCI ) \qquad ( Equation 7 )$$

where f₂ is the function that determines a ratio for increasing or decreasing the expected TRIMP (Expected_TRIMP_{w}) for the next week. The function f₂ may be modified or adjusted based on various parameters (e.g., from estimated health parameters, user feedback etc.) and may be learned using a machine learning model and/or a rule-based model, whereby the machine learning model and/or the rule-based model may at least in part utilize the estimated health parameters, user feedback, external data, or a combination thereof for generating labeling data. For example, the function f₂ may obtain labeling data from external databases that reflect improvement by the individual with respect to his or her running capacity, such as but not limited to, the individual's VO2max value. As a result, the TRIMP adjustment ratio may then be incorporated into future TCI estimations to actively monitor and/or adjust the health plan at operation 610.

By way of example, the PSI and the TCI may be determined by Equation 5 and Equation 6, respectively, for the individual, as described above. Based on the PSI and the TCI determine, the function f₂ may determine whether the expected TRIMP (Expected_TRIMP_{w}) for the upcoming week, as used in Equation 6, should be increased, decreased, or remain the same. For example, if the PSI and/or the TCI indicate that the individual has been training in an optimal range, as measured by heart rate and physical output of the individual, the expected TRIMP may be increased based on the function f₂ for the next week as the individual continues to improve their performance. Conversely, if the PSI and/or the TCI indicate that the individual has been underperforming or not motivated to complete the training plan, the expected TRIMP may be decreased based on the function f₂ for the next week as the individual continues to show regression with respect to their performance. The function f₂ may evaluate the PSI and the TCI of the individual based on the health parameters measured by the device 100 (e.g., heart rate, oxygen levels associated with the individual's VO2max, etc.)

As discussed above, the PSI and the TCI may be used to determine whether to adjust the training plan of the individual in a subsequent training period (e.g., the training plan may be adjusted for each day of the following week). The PSI and the TCI may be evaluated by their pre-defined thresholds, may be compared to pre-defined thresholds, or both to determine whether the health goal may be achieved based on the current training plan. Additionally, or alternatively, the PSI and/or the TCI may be used to determine one or more parameters, whereby the one or more parameters may be used to determine if and how the training plan may need to be adjusted. For example, the PSI or the TCI may be used to determine a parameter of the training plan and the determined parameter may be used to establish a ratio or percentage of adjustment for one or more aspects of the training plan.

The PSI and TCI values, as categorized by their pre-defined thresholds, may be evaluated at the operation 606 along with a TRIMP estimation of the initially established training plan that is estimated at operation 608. The TRIMP estimation at the operation 608 may be estimated using the above TRIMP formula. However, the TRIMP estimation for the initial health (i.e., training) plan may estimate a heart rate level based on an estimated heart rate (HR) and estimated maximum heart rate (MHR) of the individual. That is, as opposed to the TRIMP being estimated using an HR as measured or obtained as a health parameter using the device 100 during training, the HR may be estimated based upon initial user input at the operation 302 of the process 300 (e.g., age, weight, height, user estimated exercising heart rate, etc.). Similarly, the HR may be estimated using external data collected. As a result, the PSI, the TCI, and the estimated TRIMP based upon the initial health plan may all be evaluated to determine if adjustment to the health plan is necessary at the operation 606.

Based on the weekly adjustment at the operation 606 and modifications to the TRIMP at the operation 610, an adjusted training plan (i.e., health plan) may be provided to the individual at the operation 612. The training plan may be provided to the individual using the device 100 (e.g., a display of the device 100). The operation 612 may be completed or correlated to the operation 308 of the process 300 shown in FIG. 3.

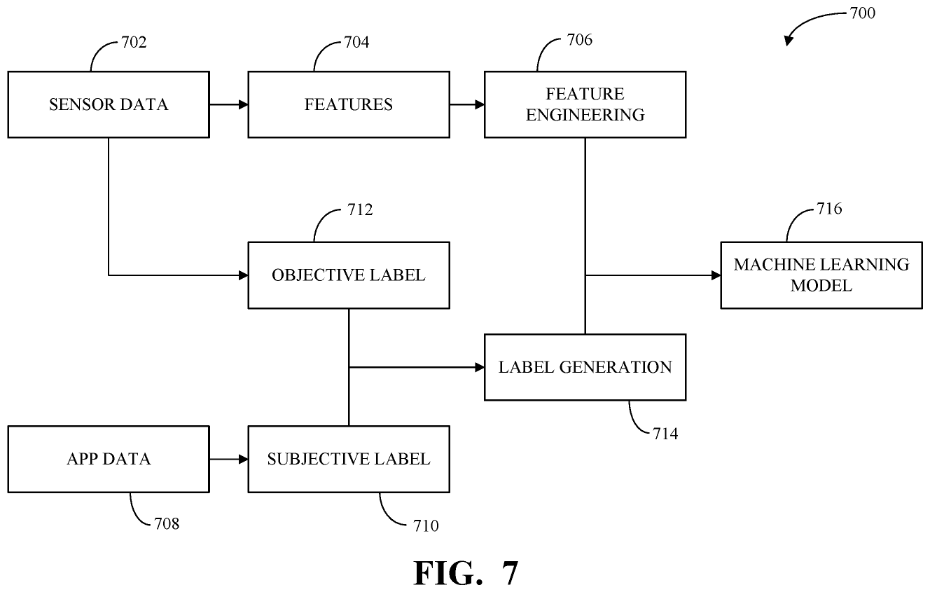

FIG. 7 illustrates a flow diagram 700 for intraweek dynamic adjustment of a health plan. The flow diagram 700 may be used interchangeably with the process 700 as detailed below. The health plan may be the health plan established at the operation 406 shown in the process 400, or similarly, the health plan established at the operation 302 or 308 of the process 300 shown in FIG. 3.

As discussed above with respect to FIG. 6, the health plan may be adjusted or modified in both short-term intervals and long-term intervals. As shown in FIG. 6, the health plan may be adjusted using one or more indices (e.g., PSI, TCI, TRIMP estimation, etc.) estimated using the one or more health parameters pertaining an individual that may be received by the device 100. Such adjustments may be completed a designated long-term interval, such as weekly. In a similar manner, adjustments to the health plan may also be done in a short-term manner based on a machine learning model established at operation 716 of FIG. 7.

While the long-term adjustment plans evaluated in FIG. 6 may focus primarily on a training status of the individual (e.g., training load, accuracy in following the training plan, etc.), the short-term adjustment plans evaluated in FIG. 7 may evaluate additional health parameters of the individual. The additional health parameters may be other physiological parameters that may or may not be directly correlated to the training status of the individual.

In particular, sensor data at operation 702 may be collected from the individual, such as by obtaining sensor date from the device 100 shown in FIG. 1. The sensor data may include measurements for the additional health parameters and may include, but are not limited to, sleep duration of the individual, interruptions during sleep of the individual, a deep-sleep ratio, heart rate variability during or outside of exercises, body temperature, or other physiological parameters. Advantageously, the individual may maintain use (i.e., wearing) of his or her device 100 throughout the day or during the night when not completing the training plan so that the device 100 may obtain further health parameter measurements. As a result, the sensor data obtained at the operation 702 may be compiled and labeled as objective data at operation 712. In doing so, the process 700 may categorize the sensor data for the upcoming machine learning model at the operation 716.

In addition to sensor data obtained at the operation 702, additional application data may be obtained at operation 708. The application data may include further data received from the individual that may not have been measured by one or more sensors of the device 100. The application data may include, but is not limited to, the user feedback provided at the operation 304 of the process 300, the initial user input received at the operation 302 of the process 300 (e.g., corresponding to the user input to set the health goal at the operation 402 shown in FIG. 4), or both. The application data may also include user input or feedback data received at another stage or operation. In certain circumstances, the device 100 may prompt the user for feedback at random intervals, whereby the feedback may vary in scope and subject matter. For example, randomly prompted questions may be provided to the user to ask for input regarding fatigue, stress levels, sleep quality, diet, injuries, or other physiological parameters. In addition, or alternatively, to randomly prompted questions, the device 100 may prompt the individual to rate their overall feeling after a training data based on one or more pre-defined scales. The rating may then be converted into a score that may estimate a fatigue level of the user for further evaluation during the process 700.

Once application data is received at the operation 708, the application data may be labeled (e.g., categorized) as subjective data at operation 710. From here, the objective labeled data from the operation 712 and the subjective labeled data from the operation 710 may compiled during label generation at operation 714. The label generation at the operation 714 may compile both the subjective and objective data to provide accurate data to the machine learning model at the operation 716 in order to train the machine learning model.

During the label generation at the operation 714, a min-max normalization function may be applied to both the subjective and objective data to avoid the impact of different or extreme ranges. The min-max normalization function may be applied as:

$$y = \frac{x - {x}_{min}}{{x}_{max} - {x}_{min}} \qquad ( Equation 8 )$$

where x is an original value of subjective or objective data, and xₘₐₓ and xₘᵢₙ are the maximum and minimal value, respectively of x. For example, the device 100 may measure the individual's sleep duration each night, which may vary by 45 minutes (e.g., 8 hours±45 minutes). By applying Equation 8 above to the measured sleep duration, the measured values may be normalized and established as a value in the range [0, 1]. Similarly, the device 100 may measure the individual's duration of time the individual wakes during sleep each night, which may vary by 10 minutes (e.g., 30 minutes±10 minutes). By applying Equation 8 above to the measured sleep wake durations, the measured values may be normalized and established as a value in the range [0, 1] similar to the sleep duration normalized values. As a result, each subjective and/or objective metric may beneficially be evaluated within a similar range [0, 1].

In normalizing the subjective and objective data, the now normalized data can be labeled as a normalized subjective metric (Sub_level) and a normalized objective metric (Obj_level), which may now both be in the range [0, 1]. Therefore, given the normalized metrics, a fatigue level (FL) may be defined as:

$$F L = Sub\_level * Obj\_level \qquad ( Equation 9 )$$

The FL may also be in the range [0, 1] and can be used as the ultimate label generated at the operation 714. For example, the device 100 may measure the sleep duration of the individual each night for a month. The measured sleep duration values may then be normalized using Equation 8 as described above and labeled as a normalized objective metric (Obj_level). Similarly, the individual may input each morning into the device 100 how many hours of sleep they had the previous night for a month. The input sleep duration values may then also be normalized using Equation 8 and labeled as a normalized subjective metric (Sub_level). The fatigue level (FL) may then be determined by Equation 9 using the Sub_level and Obj_level labels that correlate to the individual's estimated sleep durations and the measured sleep durations, respectively.

With FL now being defined (i.e., labeled), the machine learning model at the operation 716 may be trained using a regression task. Additionally, to further establish the machine learning model at the operation 716 to train the overall model, one or more algorithms may be implemented to work in conjunction with the label generation.

In one example, a deep neural network may be implemented to evaluate or interpret the FL value defined above, with the health parameters of the individual as input. Such deep neural network may be defined as:

$$F L = DNN ( TST , WT , DSR , HRV , BT ) \qquad ( Equation 10 )$$

where the fatigue level (FL) may be evaluated as a function of the deep neural network (DNN) using the sensor data obtained at the operation 702 for sleep duration (TST), wake-up during sleep or sleep interruption (WT), deep-sleep ratio (DSR), heart rate variability (HRV), and body temperature (BT). The sensor data may be defined as features of the DNN at operation 704. Once defined as features of the algorithm, a feature engineering process may be completed at operation 706 to normalize the sensor data for each defined feature. The feature engineering may use z-score for normalization of each feature to avoid the impact of different ranges among the different features. Such normalization may be:

$$z = \frac{x - u}{s} \qquad ( Equation 11 )$$

where x is the original value of each feature established at the operation 704, u is the mean value of each feature, and s is the standard deviation. The above normalization may establish common metric ranges similar to the normalization discussed above with respect to Equation 8.

Additionally, to evaluate how accurately the DNN function is operating based on the data set obtained from the established features, a loss function of the model may be a mean squared error (MSE) function, defined as the average of squared differences between the actual and predicted values for the fatigue level (FL). The MSE may be calculated as:

$$MSE = \frac{1}{N} {\sum }_{i = 1}^{N} {( F {L}_{i} - {i}_{} )}^{2} \qquad ( Equation 12 )$$

where [symbol] ᵢ is the i^{th} sample of the predicted FL and N is the total sample size.

For example, a fatigue level (FL) of the individual may be estimated using the one or more objective and/or subjective metrics from a previous week to predict the fatigue level of the individual for the upcoming week. This estimation may be based on the measured fatigue level of the individual from the previous week or separately based on the measured values of various parameters from the device 100.

The machine learning model may be established at the operation 716 using the feature engineering as described above and the label generation from the operation 714. As a result, the machine learning model implemented to train the overall model can be applied to new data during the dynamic and ongoing evaluation of the individual's health plan and health goal at the operation 412 or, similarly, at the operations 306 and/or 308.

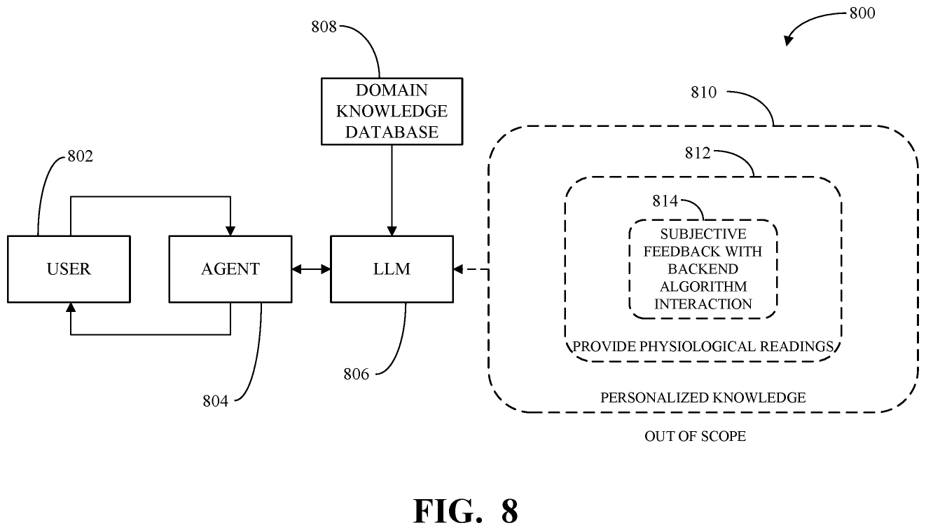

FIG. 8 is a flow diagram 800 illustrating an exemplary user-agent interaction utilizing a dynamic large language model (LLM). The LLM can serve as, for example, a health coach, a training coach or the like, providing health or training related knowledge, or answering health or training related questions input by the individual. The flow diagram 800 may be used interchangeably with the process 800 as detailed below. LLMs are a type of artificial intelligence technology that may be used in the field of natural language processing (NLP). LLMs may be particularly useful technology for building or creating a user-agent interface (i.e., “chatbot”) that may be able to generate human-like language responses to a wide range of user inputs. With respect to FIG. 8, a LLM dynamically established at operation 806 may establish such a user-agent interface between a user input at operation 802 and an agent (e.g., interface) output at operation 804. In other words, the user at the operation 802 may input questions, responses, or other data which may then be interfaced to the agent at the operation 804 established by the LLM from the operation 806, thereby allowing the agent to provide human-like responses to the user's input. The user-agent interface established between the operations 802 and 804 may be completed using the device 100 (e.g., a display of the device 100). However, the user-agent interface may not be limited to the device 100 and may be established using one or more secondary, connected devices, such as, but not limited to, a laptop, a desktop, a table, a smartphone, an additional wearable device, a voice-command device, or a combination thereof.

The user-agent interface may be limited to any desired scope of information depending on what data is used to establish the LLM. In particular, the LLM may be constrained to constrain output from the agent by defining the domain knowledge database of the LLM at operation 808. As a result, if the user input is out of scope of the domain knowledge database, the LLM may be constrained to provide a default response, thereby ensuring an inaccurate response is not provided to the user. By way of example, the domain knowledge database established may be limited to a “training” scope, whereby the user-agent interface established may focus and only provide information pertaining to general exercise training knowledge or tailored information pertaining to the individual's specific training plan and/or health goal. Beneficially, using the LLM established at the operation 806 in this manner, the user-agent interface may provide a user-centric interaction instead of a more conventional interface that may generate passive and/or generic information to the user.

Based on the defined domain knowledge database from the operation 808, the LLM may be limited to a three-layer scope based on the layers 810, 812, and 814. However, it should be noted that the scope may vary depending on a given application and couple expand to four or more layers or two or less layers. However, by way of example, a first scope layer 810 may be based on personalized knowledge. Within the first scope layer 810, a user may ask questions about generalized information pertaining to sports science that may be related to the established health plan (i.e., training plan). The following is an exemplary user-agent interaction within the first scope layer 810:
USER: What is PSI and why is it important?
COACH: Physical stress index (PSI) is a parameter that estimates the user's acute training load (ATL) compared to the chronic training load (CTL). IT is important because it reflects your current training balance. Your current PSI is 1.0, suggesting that there is stress on your body and it leads to adaptation.

A second scope layer 812 may be based on physiological readings specific to the user that may pertain to the user's continuous monitoring by the device 100. Such physiological readings may be measurements from one or more sensors of the device 100 or may be calculations or extrapolated physiological parameters based on the measured parameters. The following is an exemplary user-agent interaction within the second scope layer 812:
USER: What is my VO2max data compared to last week?
COACH: From my estimation, your current VO2max is 50 mL/(kg*min) compared to 46 mL/(kg*min) last week. It is a good sign as the increased VO2max reflects your elevated running capacity. Keep it up!

A third scope layer 814 may be based on subjective feedback provided by the user or subjective requests provided to the agent. Based on the subjective feedback provided by the user, the agent may understand the requests and may interact with an established backend algorithm. The backend algorithms may be established so that the LLM may receive the subjective input from the user and modify one or more data points within the process 300 (or sub-processes therein) based on the subjective input. Such subjective input by the user may include, but is not limited to, requesting changes to the health plan and/or health goal, requesting reminders regarding the health plan and/or the health goal (e.g., countdown to a target date), requesting a set alert for a specified task (e.g., alert to go to sleep or wake up at a specified time each day), or other subjective feedback that may be within the established scope for the LLM. The following is an exemplary user-agent interaction within the third scope layer 814:
USER: My competition has been postponed to July 1^{st}. What should I do?
AGENT: Your current racing day is Jun. 1, 2023, and I can postpone it to Jul. 1, 2023. The training plan will be adjusted accordingly to make you fully prepared for the new date. Do you want me to help you set the new racing date now?
USER: Yes, please.
AGENT: All set. The new racing date is Jul. 1, 2023. For the next week, the total training amount will be decreased slightly due to the adjustment.

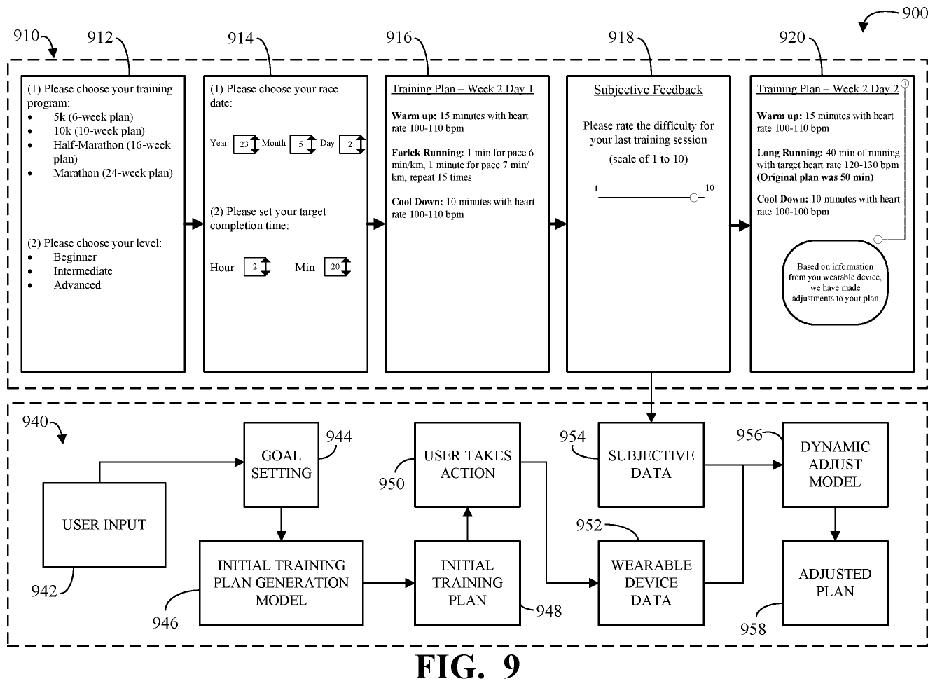

FIG. 9 illustrates exemplary user interfaces 910 and an associated flow diagram 940 for process 900. The user interfaces 910 may be established using the LLM model from FIG. 8 or may be pre-defined interfaces. The interfaces may be provided to the user via the device 100 (e.g., a screen of the device). The interfaces and flow diagram described in FIG. 9 is an exemplary process and should not be construed to in any way limit the scope of the teachings herein. It is envisioned that many variations and/or configurations may be possible based upon the present teachings.

At an initial interface 912, a user may be prompted for user input associated with operation 942 to select a training program based on a pre-defined list of training programs. For example, the user may be able to select from a training program for a 5k run, a 10k run, a half-marathon, or a full marathon. Based on the program selected, a duration of the training plan may vary. Additionally, the user input may also include selecting an initial running skill level (e.g., beginner, intermediate, or advanced). Similarly, the user may be prompted to input historical training data into the initial interface 912 so that the initial running skill level of the user may be determined more accurately. Additionally, prior performance data recorded by the device 100 may also be utilized in determined the running skill level of the user. However, the initial training programs listed by the initial interface 912 may be any exercise program and are not limited to running. For example, swimming, cycling, strength training, or other exercise programs may be provided by the initial interface 912.

Once the initial user input is provided to select a training program and a skill level, the user may be provided with a second interface 914. At the second interface 914, the user may be prompted to provide additional data, such as a race date and a target completion time for the race (i.e., pace), a target completion date for another objective of the training (e.g., weight loss training, distance swimming, strength training, etc.). Based on the data input at the second interface 914, a goal may be established at operation 944 and a training plan may be generated at operation 946. The health goal and training plan operations 944, 946 may be based on the above calculations and processes, such as those shown in processes 300, 400, 600, and 700.

After the training (i.e., health) plan has been generated at the operation 946, the initial training plan may be provided to the user at operation 948, which may correspond to a third interface 916 provided to the user through the device 100. For example, the third interface 916 may display a daily training plan, which may include one or more exercises for that day, such as, but not limited to, a specified warm-up, a specified run, and a specified cool down. The initial training plan may include one or more iterations of the third interface 916 to provide the user daily training plans for an initially set period of time (e.g., a week).

Based on the training plan provide initially provided to the user, the user may take action at operation 950 to complete the provided exercises. The operation 950 may be completed one or more times so that wearable device data is obtained by the device 100 at operation 952. As the user takes action to complete the provided training plan wearable data is obtained by the device 100, a fourth interface 918 may be provided to the user to prompt the user for subjective feedback corresponding to the current exercise plan. For example, the fourth interface 918 may prompt the user to rate or provide other feedback regarding the difficult of the most recently completed training session. This user input may correspond to the subjective data collection at operation 954.

Based upon the wearable device data obtained by the device 100, such one or more health parameters of the user, and the subjective data input by the user at operation 954, the dynamic adjustment model at operation 954 may be completed. Such adjustment model may be based on the operation 306 of the process 300, which may include dynamic evaluation and modification to the training plan based on the processes 400, 600, and 700, as discussed in further detail above. The evaluation completed based on the dynamic adjustment model may determine whether modifications to the current training plan are needed to reach the established health goal set at the operation 944. If the model determines changers to the training plan are necessary, an adjusted training plan may be generated and provided to the user at operation 958, which may correspond to a fifth interface 920.

As illustrated in FIG. 9, the fifth interface 920 may provide the user with a modified training plan and may indicate to the user that the initially established training plan has been modified based upon data collected. This process may be actively completed one or more times throughout the training plan to aid the user in reaching their established health goal. In doing so, the process 900 may actively monitor the user and health parameters of the user while also requesting subjective user input to evaluate the training program and minimize the risk of injury, all while maximizing the likelihood of the individual achieving their established health goal.

It should also be noted that while evaluation and modification of the training plan associated with the health goal has been discussed in detail, such evaluation and modification may also be completed with respect to the initially established health goal. That is, the processes 300, 400, 600, and 700 may also include active evaluation of the health goal itself. For example, if evaluation of the health plan determines that no adjustments to the health plan are possible to increase the likelihood the user achieving his or her initially established health goal, the user may be notified that the health goal is unattainable. As a result, the user may be prompted modify their health goal. Similarly, if an initially set health goal by the user is evaluated and determined to be unattainable regardless of the training program implemented, the user may be prompted to input an alternative health plan. Therefore, one skilled in the art may glean from the present teachings that additional customization and flexibility within the health goal and/or the associated health plan may be possible.

Technical specialists skilled in the art should understand that the implementations in this disclosure may be implemented as methods, systems, or computer program products. Therefore, this disclosure may be implemented in forms of a complete hardware implementation, a complete software implementation, and a combination of software and hardware implementation. Further, this disclosure may be embodied as a form of one or more computer program products which are embodied as computer executable program codes in computer writable storage media (including but not limited to disk storage and optical storage).

This disclosure is described in accordance with the methods, devices (systems), and flowcharts and/or block diagrams of computer program products of the implementations, which should be comprehended as each flow and/or block of the flowcharts and/or block diagrams implemented by computer program instructions, and the combinations of flows and/or blocks in the flowcharts and/or block diagrams. The computer program instructions therein may be provided to generic computers, special-purpose computers, embedded computers or other processors of programmable data processing devices to produce a machine, wherein the instructions executed by the computers or the other processors of programmable data processing devices produce an apparatus for implementing the functions designated by one or more flows in the flowcharts and/or one or more blocks in the block diagrams.

The computer program instructions may be also stored in a computer readable storage which is able to boot a computer or other programmable data processing device to a specific work mode, wherein the instructions stored in the computer readable storage produce a manufactured product containing the instruction devices which implements the functions designated by one or more flows in the flowcharts and/or one or more blocks in the block diagrams.

The computer program instructions may also be loaded to a computer or another programmable data processing device to execute a series of operating procedures in the computer or the other programmable data processing device to produce a process implemented by the computer, whereby the computer program instructions executed in the computer or the other programmable data processing device provide the operating procedures for the functions designated by one or more flows in the flowcharts and/or one or more blocks in the block diagrams.

Apparently, the technical specialists skilled in the art may perform any variation and/or modification to this disclosure by the principles and within the scope of this disclosure. Therefore, if the variations and modifications herein are within the scope of the claims and other equivalent techniques herein, this disclosure intends to include the variations and modifications thereof.

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising”, and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. The terms “at least one of A or B,” “at least one of A and B,” “one or more of A or B,” “A and/or B” used herein mean “A”, or “B” or “A and B”.

While the disclosure has been described in connection with certain embodiments or implementations, it is to be understood that the disclosure is not to be limited to the disclosed embodiments but, on the contrary, is intended to cover various modifications and equivalent arrangements included within the scope of the appended claims, which scope is to be accorded the broadest interpretation so as to encompass all such modifications and equivalent structures as is permitted under the law.

What is claimed is:

1. A method of dynamically monitoring a health goal using a wearable device, comprising:
receiving, by a processor, user input associated with the wearable device worn by an individual to determine the health goal for the individual and a health plan associated with the health goal for the individual to reach the health goal;
obtaining, by the processor, health parameters associated with the individual from the wearable device and user feedback from the individual based on a physical condition of the individual associated with the health plan;
evaluating, by the processor based on the health parameters and the user feedback, whether the health goal will be successfully reached by the individual following the health plan; and
responsive to determining that the health goal will not be successfully reached by the individual following the health plan, dynamically adjusting the health plan based upon the health parameters and the user feedback to determine a modified health plan, wherein the modified health plan is provided to the individual.

2. The method of claim 1, wherein the user input includes at least one of the following items: a target distance, a level of skill, or a target date, and wherein at least one of the target distance, the level of skill, or the target date is evaluated to establish the health goal.

3. The method of claim 2, wherein the user input further includes a current performance metric of the individual that is compared to the health goal to determine a gap between the current performance metric and the health goal, and wherein the gap is evaluated to create the health plan.

4. The method of claim 1, wherein evaluating, based on the health parameters and the user feedback, whether the health goal will be successfully reached by the individual following the health plan further comprises:
estimating at least one of a physical stress index or a training capability index based on the health parameters; and
evaluating at least one of the physical stress index or the training capability index to determine whether the health goal will be successfully reached.

5. The method of claim 4, wherein the health parameters include an exercising heart rate of the individual measured by one or more sensors of the wearable device and obtained by the processor to estimate a training impulse value, and
wherein estimating at least one of the physical stress index or the training capability index based on the health parameters, further comprises:
estimating the physical stress index by comparing an accumulation of the training impulse value in a first duration of time to an accumulation of the training impulse value in a second duration of time, wherein the first duration of time is shorter than the second duration of time, or
estimating the training capability index by comparing the training impulse value to an expected training impulse value that is estimated based on the user input to determine the health plan.

6. The method of claim 4, wherein a training impulse adjustment ratio is calculated, by the processor, based upon at least one of the physical stress index or the training capability index, and wherein the modified health plan is determined based upon the training impulse adjustment ratio.

7. The method of claim 1, wherein evaluating, by the processor based on the health parameters and the user feedback, whether the health goal will be successfully reached by the individual following the health plan further comprises:
determining, by the processor, a machine learning model configured to evaluate whether the health goal will be successfully reached by the individual following the health plan;
training, by the processor, the machine learning model with the health parameters and the user feedback; and
evaluating, by the processor using the machine learning model, whether the health goal will be successfully reached by the individual following the health plan.

8. The method of claim 7, wherein the modified health plan is determined by the machine learning model configured to evaluate whether the health goal will be successfully reached by the individual following the health.

9. The method of claim 4, further comprising:
determining, by the processor, a machine learning model configured to dynamically adjust the health plan based upon the health parameters and the user feedback to determine the modified plan; and
training, by the processor, the machine learning model with the health parameters and the user feedback, wherein the modified health plan is determined based on the dynamic adjustments to the health plan by the machine learning model.

10. The method of claim 1, further comprising:
determining, by the processor, a machine learning model configured to estimate a fatigue level of the individual; and
training, by the processor, the machine learning model with the health parameters and the user feedback, wherein the health parameters comprise physiological parameters associated with the individual and exercise performance parameters associated with completed exercise tasks of the individual, and wherein the modified health plan is determined based upon the fatigue level estimated.

11. The method of claim 1, further comprising:
dynamically establishing, by the processor, a large language model (LLM) using the health parameters associated with the individual, the user feedback from the individual and data from an external database; and
establishing, by the processor, a user-agent interface configured to obtain the user input and output information based upon the user input, wherein the user-agent interface is established based upon the large language model (LLM).

12. An apparatus for dynamically monitoring a health goal using a wearable device, the apparatus comprising:
a non-transitory memory; and
a processor configured to execute instructions stored in the non-transitory memory to:
receive user input associated with the wearable device worn by an individual to determine the health goal for the individual and a health plan associated with the health goal for the individual to reach the health goal;
obtain health parameters associated with the individual from the wearable device and user feedback from the individual based a physical condition of the individual associated with the health plan;
evaluate, based on the health parameters and the user feedback, whether the health goal will be successfully reached by the individual following the health plan; and
responsive to determining that the health goal will not be successfully reached by the individual following the health plan, dynamically adjust the health plan based upon the health parameters and the user feedback to determine a modified health plan and provide the modified health plan to the individual.

13. The apparatus of claim 12, wherein the instructions to evaluate, based on the health parameters and the user feedback, whether the health goal will be successfully reached by the individual following the health plan further comprise instructions to:
estimate at least one of a physical stress index and a training capability index based on the health parameters; and
evaluate at least one of the physical stress index or the training capability index to determine whether the health goal will be successfully reached.

14. The apparatus of claim 13, wherein the health parameters include an exercising heart rate of the individual measured by one or more sensors of the wearable device and obtained by the processor to estimate a training impulse value, and
wherein estimating at least one of the physical stress index or the training capability index based on the health parameters further comprises:
estimating the physical stress index by comparing an accumulation of the training impulse value in a first duration of time to an accumulation of the training impulse value in a second duration of time, wherein the first duration of time is shorter than the second duration of time; or
estimating the training capability index by comparing the training impulse value to an expected training impulse value that is estimated based on the user input to determine the health plan.

15. The apparatus of claim 13, wherein a training impulse adjustment ratio is calculated based upon at least one of the physical stress index or the training capability index, and wherein the modified health plan is created based upon the training impulse adjustment ratio.

16. The method of claim 12, wherein the instructions to evaluate, based on the health parameters and the user feedback, whether the health goal will be successfully reached by the individual following the health plan further comprise instructions to:
determine a machine learning model configured to evaluate whether the health goal will be successfully reached by the individual following the health plan;
train the machine learning model with the health parameters and the user feedback; and
evaluate, using the machine learning model, whether the health goal will be successfully reached by the individual following the health plan.

17. The method of claim 16, wherein the modified health plan is determined by the machine learning model configured to evaluate whether the health goal will be successfully reached by the individual following the health.

18. The method of claim 12, wherein the instructions further comprise instructions to:
determine a machine learning model configured to dynamically adjust the health plan based upon the health parameters and the user feedback to determine the modified plan; and
train, the machine learning model the health parameters and the user feedback, wherein the modified health plan is determined based on the dynamic adjustments to the health plan by the machine learning model.

19. The apparatus of claim 12, wherein the instructions further comprise instructions to:
dynamically establish a large language model (LLM) using the health parameters associated with the individual, the user feedback from the individual, and data from an external database; and
establish a user-agent interface configured to obtain the user input and output information based upon the user input, wherein the user-agent interface is established based upon the large language model (LLM).

20. A non-transitory computer-readable storage medium configured to store computer programs for dynamically monitoring a health goal using a wearable device, the computer programs comprising instructions executable by a processor to:
receive user input associated with the wearable device worn by an individual to determine the health goal for the individual and a health plan associated with the health goal for the individual to reach the health goal;
obtain health parameters associated with the individual from the wearable device and user feedback from the individual based on a physical condition of the individual associated with the health plan;
evaluate, based on the health parameters and the user feedback, whether the health goal will be successfully reached by the individual following the health plan; and
responsive to determining that the health goal will not be successfully reached by the individual following the health plan, dynamically adjust the health plan based upon the health parameters and the user feedback to determine a modified health plan and provide the modified health plan to the individual.
